(19) United States
(12) Patent Application Publication
(10) Pub. No.: US 2013/0189724 A1
(43) Pub. Date: Jul. 25, 2013

(54) USE OF AN ADAPTIVE CHEMICALLY REACTIVE PLASMA FOR PRODUCTION OF MICROBIAL DERIVED MATERIALS

(71) Applicant: C-Tech LLC, Las Vegas, NV (US)

(72) Inventors: Timothy E. Meehan, Richland, WA (US); Leslie J. Peterson, Kennewick, WA (US); Mark R. Maier, Murfreesboro, TN (US)

(73) Assignee: C-TECH LLC, Las Vegas, NV (US)

(21) Appl. No.: 13/791,703

(22) Filed: Mar. 8, 2013

Related U.S. Application Data
(63) Continuation-in-part of application No. 12/792,545, filed on Jun. 2, 2010.
(60) Provisional application No. 61/169,201, filed on Sep. 1, 2009.

(51) Int. Cl. C12M 1/00

(52) U.S. Cl. CPC C12M 29/00
USPC 435/29; 435/161; 435/160; 435/132; 435/106; 435/286.1; 435/289.1; 435/167

(57) ABSTRACT

A carbonaceous feedstock can be converted into a chemical product having a higher commercial value, by introducing the carbonaceous feedstock into a plasma torch under conditions selected to generate a synthetic gas mixture having a tailored composition, and then introducing the synthetic gas mixture into a microbial digester configured to convert the synthetic gas mixture into the chemical product. The composition of the synthetic gas mixture produced by the plasma torch can be quickly modified to yield an optimal quality and quantity required by the microbial digester, and the quantity and stoichiometric ratio can be quickly varied if the quantity or composition of the synthetic gas mixture being provided is not appropriate for the microbial digester, enabling greater efficiency to be achieved as compared to systems where the quality and quantity of the synthetic gas mixture cannot be easily changed.

### RELATED APPLICATIONS

This application is a continuation-in-part (CIP) of a prior copending application, Ser. No. 12/792,545, filed Jun. 2, 2010, which is based on a prior provisional application, Ser. No. 61/169,201, filed on Sep. 1, 2009, the benefit of the filing dates of which is hereby claimed under 35 U.S.C. §120 and 35 U.S.C. §119(e).

### BACKGROUND

Synthetic gas mixtures can be used in microbial digesters to generate different chemical products. A common synthetic gas mixture primarily composed of CO and H₂ is frequently referred to as syngas. It should be recognized that the term synthetic gas mixture as used herein is broader than the term syngas, as syngas refers to a specific synthetic gas mixture whose primary components are CO and H₂, while the broader term synthetic gas mixture encompasses non-naturally occurring (i.e., man-made) gas mixtures which include additional gases (i.e., gases in addition to CO and H₂), as well as synthetic gas mixtures that include little or no CO and/or H₂. Unfortunately, most industrial processes for generating a desired synthetic gas mixture in quantity cannot readily change a quantity or quality (i.e., a targeted stoichiometric ratio of the components in the synthetic gas) of the synthetic gas quickly, to adapt to changing conditions, which can exist in microbial digesters or microbial production based processes. Also, current synthetic gas production processes have difficulty achieving high ratios of CO over H₂; a stoichiometry favored by many microbial systems. Thus, the microbial digester does not receive a synthetic gas mixture in an optimal quality or quantity to maximize production of the desired product. An overall excess of synthetic gas can be provided to make up for a particular deficient component quantity, but providing excess synthetic gas leads to system inefficiencies and wasted resources from the over production of the other synthetic gas mixture components.

It would be desirable to provide a system capable of efficiently providing synthetic gas mixtures in quality and quantity that match the needs of the microbial digester/process at a given time.

### SUMMARY

This application specifically incorporates by reference the disclosures and drawings of each patent application and issued patent identified above as a related application. Furthermore, this application specifically incorporates by reference the disclosures and drawings of U.S. Pat. Nos. 7,285,402 and 5,173,429, which disclose the generation of chemical products using microbial digestion.

In accord with the concepts disclosed herein, an exemplary method is defined for converting any carbonaceous feedstock into a tailored, synthetic gas mixture, using CO₂, steam, N₂ or any other ionizing input gas, such that the tailored synthetic gas mixture will comprise the required components, and the input gas will be used as both a chemical reactant and as the gaseous fluid that is ionized to produce a thermal plasma. In this method, ionized gases produced by a plasma torch and an appropriate feedstock are mixed in a reaction vessel to produce a synthetic gas mixture that meets a desired stoichiometric ratio (the desired stoichiometric ratio being matched to a particular microbial digester/process). The synthetic gas mixture is then injected into a microbial digester, at the appropriate temperature and pressure, to convert the synthetic gas mixture into a higher value commercial product. The separation and purification of the product material from the microbial environment is conducted through conventional means of separation and purification to achieve the desired purification.

The term synthetic gas mixture encompasses gaseous mixtures including one or more of the following components, in any combination: hydrogen, carbon monoxide, carbon dioxide, nitrogen, and hydrogen sulfide (such gaseous components being exemplary, rather than limiting). The term “higher alcohols” refers to alcohols with two or more carbon atoms, examples of which include ethanol, butanol, and hexanol—without any implied limitation. The term “plasma torch,” as used herein and in the claims that follow, is intended to encompass ionized gas plasma generating systems, such as Inductively Coupled Plasma, Transferred Arc DC Plasma, and Non-Transferred Arc DC Plasma (such systems being exemplary, rather than limiting). The term “plasma reactor,” as used herein and in the claims that follow, is intended to refer to a combination of a plasma torch and a reaction vessel with which the plasma torch is used. The term “quality,” as used herein and in the claims that follow, in reference to a synthetic gas mixture, is intended to refer to how closely the quantity ratio of the components of a synthetic gas mixture matches the requirements of a microbial digester for producing a desired chemical product. The term “tailored synthetic gas mixture,” as used herein and in the claims that follow, is intended to refer to a synthetic gas whose composition has been tailored to meet the needs of a downstream microbial digester that uses the tailored synthetic gas mixture as a feedstock.

Significantly, this method has the advantage of being able to rapidly adjust both the quantity and quality (i.e., the composition) of the synthetic gas mixture being produced in response to changes in the downstream microbial digesters and bio-reactors. With respect to the quantity of the synthetic gas mixture, by analyzing the effluent from the digester/bio-reactor, one can determine if surplus synthetic gas mixture is being introduced into the digester, beyond that needed for optimal conditions. Such a surplus of the synthetic gas mixture represents wasted power, input gas, and feedstock used to create the synthetic gas mixture. Reducing the output of the plasma reactor to match the demand required in the digester will increase system efficiency. With respect to the quality of the synthetic gas mixture (i.e., the composition and stoichiometric ratio of the components of the synthetic gas mixture), by analyzing the conditions in the digester, one can determine if the ratio needs to be changed to provide optimal conditions in the digester. Providing the digester with a synthetic gas mixture that is too “rich” in a certain component similarly represents a systemic inefficiency that can be reduced. Note that as conditions in the digester change, the optimal stoichiometric ratio of the components of the synthetic gas mixture will likely also change. Optimal stoichiometric ratios can be determined by empirical data, or by routinely varying the ratio and monitoring the conditions and quality of the product exiting the digesters to determine an optimal synthetic gas mixture ratio. It should also be noted that the operating characteristic of the plasma torch can be varied as needed to produce a synthetic gas mixture in desired quantity and quality. Thus, changes in the consistency of the feedstock can be accommodated by changing the plasma torch operating conditions (and by changing the input gases introduced into the plasma torch) to enable the production of a synthetic gas mixture in the desired quantity and quality.

The exemplary method employs a plasma generator, a variable gas supply system, a variable power supply connected to energize the plasma generator, a reaction vessel having an inlet adapted to receive a thermal plasma produced by the plasma generator and an outlet from which a product is collected, and a variable carbonaceous feed supply system adapted to inject the required feed into the reaction vessel.

Any gas that when ionized generates a plasma (argon, CO, CO₂, O₂, steam, N₂, CH4, etc.) is supplied to the plasma generator, and ionized to produce the thermal plasma that is injected into the reaction vessel. The carbonaceous feedstock is injected into the reaction vessel to react with the ionized gas. In this process, the plasma gas acts not only as a thermal source to produce the thermal plasma that provides energy to drive an endothermic reaction, but is also a reactant in the reaction, and would be selected based on the required composition of the synthetic gas mixture.

A plurality of different reactions can be carried out using a plasma gas that reacts with a carbonaceous feedstock. For example, to achieve a CO rich synthetic gas mixture using CO₂ plasma, placing carbon in the reaction vessel produces CO with relatively little or no hydrogen. Adding carbon and water to the reaction vessel produces a H₂:CO synthetic gas mixture based on the stoichiometric ratio of water added. The same process can be used with steam plasma. If the microbes selected produce amino acids, using nitrogen plasma to provide the nitrogen and thermal energy to convert carbon and water into H₂ and CO can produce the desired stoichiometric ratio required by the microbial digester. The reactant feed can be a gas, a liquid, a solid or any combination thereof. Any ash, sulfur or particulate residue from using a coal or biomass feed is filtered out, leaving the tailored synthetic gas mixture.

All or part of the feedstock material can be mixed with the plasma before ionization by the plasma torch, instead of separately injecting the feedstock material into the reaction vessel. A portion of the (non-ionized) gas can be injected into the reaction vessel along with the feedstock material, the additional gas being supplied in sufficient quantity to completely react with the feedstock material.

In the concepts disclosed herein, a plasma generator is employed. A control device is provided to selectively control the gas supply system, the power supply for the plasma torch, and the feedstock supply system. Optimal efficiency of the plasma torch can be achieved by selectively varying either the gas supply system and/or the current supplied by the power supply to energize the plasma torch. The control system preferably includes a processor coupled with at least one sensor that measures torch efficiency. The processor for the control device can be programmed to adjust the gas flow rate and the power level automatically to optimize the torch efficiency.

Alternatively (or additionally), the control system can be configured to optimize the composition of the synthetic gas mixture by selectively varying the power level, the gas flow rate, and/or the carbonaceous feed rate. The flow rates of the gas required to optimize the composition of the synthetic gas mixture can be based upon the reaction between the ionized plasma gas produced by the plasma torch, the feedstock material, and any non-ionized gas injected into the reaction vessel. Once these levels are determined, the control device can be employed to automatically vary the flow rates of the feedstock material and any additional non-ionized gas flow into the reaction vessel to maximize the yield of the synthetic gas mixture from the reaction vessel.

A feedback sensor is preferably disposed at the outlet of the reaction vessel for monitoring the composition of the synthetic gas mixture exiting the reaction vessel. This sensor provides data to the processor, which through an appropriate software program, is used to monitor and automatically vary the feedstock material feed rate and the non-ionized gas flow rate into the reaction vessel to match the composition of the synthetic gas mixture produced to the composition of the synthetic gas mixture required by the microbial digester.

Means are provided for monitoring one or more conditions in the microbial digester and include another feedback sensor that is disposed proximate the exhaust of the microbial digester, and which is used to monitor any components of the synthetic gas mixture that are not consumed during the microbial digestion (exhaust gas). The amount and composition of the exhaust gas is an indication of the microbial digestion rate and efficiency, which may vary based on microbial population and health. Too much or too little of any particular component of the synthetic gas mixture exiting the microbial digester provides data to the processor to be used to vary the input feeds to achieve the correct amount and composition of the synthetic gas mixture required in the microbial digester. Analysis of exhaust gas rates and composition may also indicate the need for adjustment of microbial tank environmental conditions and/or nutrients; allowing the operator to selectively adjust production of the tailored synthetic gas mixture on-the-fly (by changing a quantity or composition of the synthetic gas mixture), or to change the microbial digester environment.

The processor is preferably programmed to selectively give priority to optimizing the operating efficiency of the plasma torch, the yield of the synthetic gas mixture from the reaction vessel, or the product yield from the microbial digester. The operator can elect which of these efficiencies will have priority.

Another aspect of the concepts disclosed herein is directed to a system utilizing the plasma gas to serve both as a thermal source to provide energy to drive the endothermic reaction, and as a reactant in the process. Such a system includes elements that function in a manner generally consistent with the steps of the methods discussed above.

Further, increases in the synthetic gas mixture yield can be obtained by adjusting at least one of the power level applied to the plasma torch, the flow rate of the gaseous fluid into the plasma torch, and the flow rate of the gaseous fluid into the reaction vessel. Changing the power level applied to the plasma torch or the flow rate of the gaseous fluid into the plasma torch will affect the plasma torch efficiency. Depending on the preferences of the operator, priority can be given to optimizing the operation of the plasma torch, optimizing the synthetic gas mixture yield of the reaction vessel, the product yield of the microbial digester or some combination thereof.

Once the composition of the synthetic gas mixture is optimized, it is drawn into the microbial digester to produce the desired target material (i.e., a chemical product such as ethanol, butanol, or acetate). Cooling or heating of the synthetic gas mixture may be required to fit the environmental parameters of the microbial digester. Any extraneous component in the synthetic gas mixture such as ash, silica, SOₓ, NOₓ, hydrogen sulfide, etc., can be filtered and/or scrubbed using conventional means for removing that component.

The microbial digester introduces the clean synthetic gas mixture into the microbial digesting chamber or column, where the microbes or group of microbes produce targeted materials from the synthetic gas mixture. The parameters of the synthetic gas mixture, (i.e., flow rate, pressure and temperature) are selected using parameters determined to optimize material production within the digester environment. Any food or water supplied to the microbial digester is also added to the digester, using parameters selected to promote production of the targeted materials. The conditions in the digester are monitored for microbial growth, temperature, pH, and other environmental parameters associated with the microbial growth. Where the composition of the synthetic gas mixture can be varied to produce a synthetic gas mixture exhibiting a desired environmental parameter, such as pH and CO₂ concentration, the operator can vary the composition of the synthetic gas mixture through the feedback loop discussed above to obtain the desired conditions within the digester.

Another aspect of the concepts disclosed herein is a technique for reducing or eliminating CO₂ emissions in industrial processes, while generating useful chemical products. As generally discussed above, CO₂ and a carbonaceous feedstock can be reformed by a plasma torch to generate a synthetic gas mixture, which can be used to produce chemical products such as ethanol or acetate in bio-reactors. This process requires modest capital investment in the plasma reactor and digester. Operating costs include the acquisition of a carbonaceous feedstock (which can be a low value feedstock, such as carbon waste or biomass) and the electrical power required to operate the torch. Offsetting those capital and operating costs are the value of the chemical product produced by the digester, and the reduction or elimination in expenses associated with managing CO₂ emissions. Generally as discussed above, the operation of the plasma torch can be manipulated to match the quality and quantity of the synthetic gas mixture being produced to meet the needs of the downstream digester/bio-reactor. Particularly if trading in carbon credits matures, there may be significant financial incentives to eliminate CO₂ emissions using this technique.

This Summary has been provided to introduce a few concepts in a simplified form that are further described in detail below in the Description. However, this Summary is not intended to identify key or essential features of the claimed subject matter, nor is it intended to be used as an aid in determining the scope of the claimed subject matter.

### DRAWINGS

Various aspects and attendant advantages of one or more exemplary embodiments and modifications thereto will become more readily appreciated as the same becomes better understood by reference to the following detailed description, when taken in conjunction with the accompanying drawings, wherein:

FIG. 1 is a simplified process flow diagram for a Chemical Conversion Reaction (CCR) process implemented in a reaction vessel with a plasma torch, in accord with the concepts disclosed herein;

FIG. 2 is a simplified process flow diagram for a CCR process that converts a carbonaceous feedstock into a tailored synthetic gas mixture, illustrating how heat is recovered from the hot synthetic gas mixture to further enhance the overall system efficiency;

FIG. 3 is a process flow diagram for a process that converts a synthetic gas mixture having a tailored composition into a targeted material through aerobic or anaerobic digestion, and subsequent purification of the targeted material produced by the microbes;

FIG. 4 is a process flow diagram for a system that converts a carbonaceous material into a synthetic gas mixture having a tailored composition using a plasma gas, introduces the synthetic gas mixture into a microbial digester, then separates and purifies the chemical product generated in the digester, and recycles processing waste created by the digestion and purification steps, where the system includes feedback loops analyzing the synthetic gas mixture and digester outputs for enabling the system to be optimized for efficiency;

FIG. 5 is a process flow diagram for a system that converts biomass, such as straw, into a synthetic gas mixture using a plasma gas, introduces the synthetic gas mixture into a microbial digester, then separates and purifies the chemical product generated in the digester, and recycles processing waste created by the digestion and purification steps, where the system includes feedback loops for analyzing the synthetic gas mixture and digester outputs for enabling the system to be optimized for efficiency, and a water gas shifting component;

FIG. 6 is a process flow diagram for a system that converts biomass such as straw into a synthetic gas mixture using a plasma gas, introduces the synthetic gas mixture into a microbial digester, then separates and purifies the chemical product generated in the digester, and recycles processing waste created by the digestion and purification steps, where the system includes feedback loops analyzing the synthetic gas mixture and digester outputs for enabling the system to be optimized for efficiency;

FIG. 7 is a functional block diagram for a basic system for implementing the concepts disclosed herein;

FIG. 8 is a flow chart showing exemplary steps for implementing the concepts disclosed herein; and

FIG. 9 is an exemplary functional block diagram and mass energy balance for an embodiment that simultaneously reduces CO₂ emissions from a CO₂ generating facility and produces ethanol.

### DESCRIPTION

### Figures and Disclosed Embodiments Are Not Limiting

Exemplary embodiments are illustrated in referenced Figures of the drawings. It is intended that the embodiments and Figures disclosed herein are to be considered illustrative rather than restrictive. No limitation on the scope of the technology and of the claims that follow is to be imputed to the examples shown in the drawings and discussed herein. Further, it should be understood that any feature of one embodiment disclosed herein can be combined with one or more features of any other embodiment that is disclosed, unless otherwise indicated.

### General Overview of the Concepts Disclosed Herein

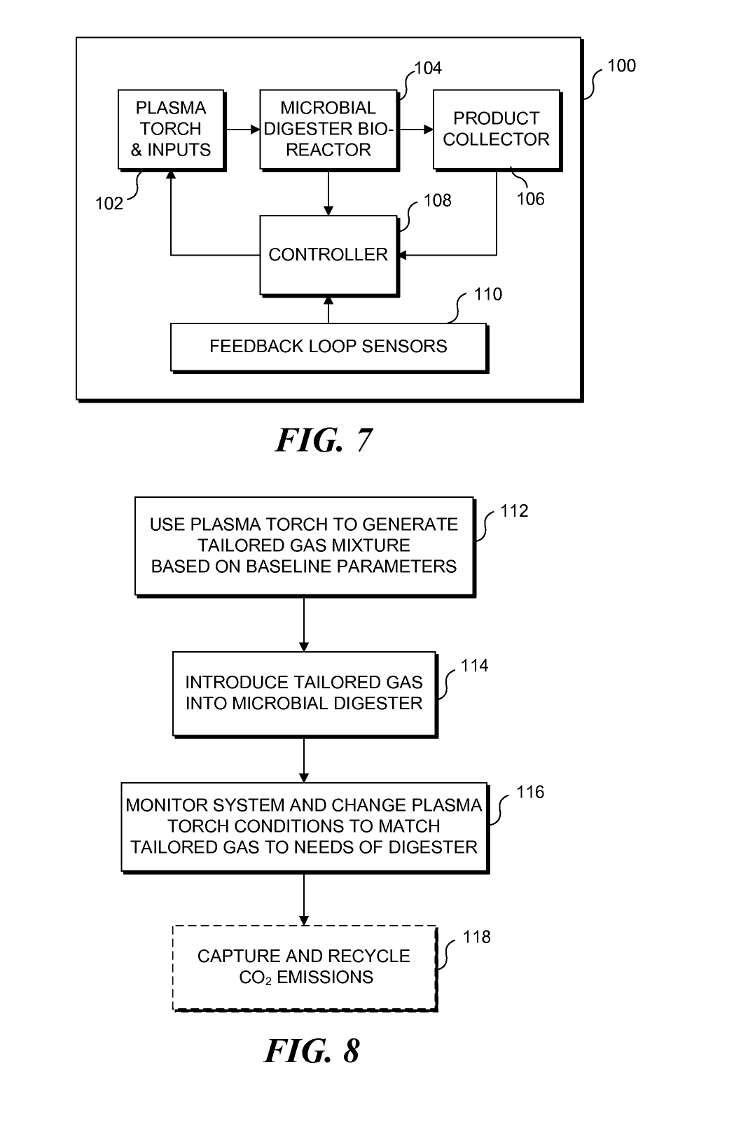

FIG. 7 is a functional block diagram for a basic system 100 for implementing the concepts disclosed herein. The basic system includes a plasma torch and inputs 102, a microbial digester/bio-reactor 104, a product collector component 106, a controller 108, and feedback loop sensors 110 (which are optional, but not required). The plasma torch and inputs includes elements required for operation of the plasma torch to generate a synthetic gas mixture having a tailored composition, including gaseous inputs, carbonaceous feedstocks, and a power source. The specific locations of various feedback loop sensors (discussed in detail elsewhere herein) that are used by the controller to ensure that the quantity and quality of the synthetic gas mixture produced in the plasma torch correspond to needs in the digester are not specifically shown. The controller can be implemented using a custom circuit (i.e., a hardware implementation, such as an application specific integrated circuit or other logic circuit), or a processor executing machine instructions stored in a memory (i.e., a software implementation, where the software is executed by a computing device, such as a desktop or laptop computer).

FIG. 8 is a flow chart for exemplary steps for implementing the concepts disclosed herein. In a first step 112, a plasma torch is used to generate a baseline quantity and quality of a synthetic gas mixture from a relatively low value carbonaceous feedstock. The baseline values are based on empirically determined values for initial conditions in the microbial digester bio-reactor. In a second step 114, the synthetic gas mixture is introduced into the microbial digester (gas conditioning, particularly gas cooling, may be required). In a third step 116, the overall system is monitored to determine if the baseline values meet the needs of the microbial digester, and when a quantity and quality of the synthetic gas mixture needs to be changed to meet the needs of the microbial digester, the plasma torch operating conditions are modified to change the quantity and quality of the synthetic gas mixture to correspond to the needs of the microbial digester. In an optional fourth step 118, CO₂ emission from the system are captured and recycled. Such CO₂ emissions can be generated by combusting a fuel to generate system power, and can also be emitted from the digester. The CO₂ emissions are recycled by capturing the CO₂ and using a feedstock in the plasma reactor to generate the tailored synthetic gas mixture.

Note that the digester can utilize a single microbe, or a consortium of different microbes, and such microbes can be naturally occurring or genetically engineered/modified. Further, multiple digesters can be employed, in series or in parallel.

Many microbial processes do not work to produce a product by using a single isolated microbe in the digester, but by using several types of microbes living in the digester, often all in the same microbial family, which collectively generate the desired product (such a mixture of microbes is typically referred to as a “consortium”). An example of this is the current production of methane in wastewater treatment plants. In those digester tanks reside several hundred distinctly different microbes in the methanogenic family, all working to digest the sludge and produce methane.

### Advantages of the Concepts Disclosed Herein

Significantly, this method has the advantage of being able to rapidly adjust both the quantity and quality (i.e., composition) of the synthetic gas mixture being produced in response to changes in the downstream microbial digesters. With respect to the quantity of the synthetic gas mixture, by analyzing the effluent from the digester, one can determine if a surplus of the synthetic gas mixture is being introduced into the digester, beyond that needed for optimal conditions. Such surplus synthetic gas mixture represents wasted power, input gas, and carbonaceous feedstock used to create the synthetic gas mixture. Reducing the output of the plasma reactor to match the demand required in the digester will increase system efficiency. With respect to the quality of the synthetic gas mixture (i.e., the stoichiometric ratio of the desire components of the synthetic gas), by analyzing the conditions in the digester, one can determine if the ratio of gaseous components, or the composition of the synthetic gas mixture, needs to be changed to provide optimal conditions in the digester. Providing the digester a synthetic gas mixture including a surplus of any gaseous component represents a systemic inefficiency that can be reduced. Note that as conditions in the digester change, the optimal stoichiometric ratio of the components of the synthetic gas mixture will likely also change. Optimal stoichiometric ratios can be determined by empirical data, or by routinely varying the ratio and monitoring the conditions and quality of the product exiting the digesters to determine an optimal synthetic gas mixture ratio (composition). It should also be noted that the operating characteristic of the plasma torch can be varied as needed to produce a synthetic gas mixture in a desired quantity and quality. Thus, changes in the carbonaceous feedstock can be accommodated by changing the plasma torch operating conditions (and by changing the input gases introduced into the plasma torch) to enable synthetic gas mixture in the desired quantity and quality to be produced (i.e., so that the composition and quantity of the synthetic gas mixture can be tailored to the needs of the digester).

### Production of a Synthetic Gas Mixture Having a Tailored Composition

Plasma torches are well suited to provide the thermal energy required to drive many chemical reactions, for example, to produce a synthetic gas mixture having a tailored composition (components of such a synthetic gas mixture can include, but are not limited to, carbon monoxide (CO) and hydrogen (H₂)). The successful use of a chemically reactive plasma serving both as a reactant and as a source of heat to drive endothermic reactions for industrial applications requires good overall efficiency, with respect to both the operation of the plasma torch and the process that produces the desired product, to attain favorable process economics. Chemically reactive plasmas can tailor a synthetic gas mixture composition using the plasma gas as well as reactant gases to achieve a desired stoichiometric composition, and the desired stoichiometric composition can be varied by changing the plasma or reactant gas compositions and quantities.

Existing gasification technologies are typically designed for production of a synthetic gas mixture having a specific stoichiometric composition that cannot be readily varied, in contrast to the ability of a plasma reactor to provide synthetic gas mixtures whose compositions can be readily varied using the same base equipment configuration. An important aspect of the concepts disclosed herein is the ability to produce a tailored synthetic gas mixture having a specific stoichiometry without the addition of undesired elements. A further important aspect of the concepts disclosed herein is the ability to operate at variable power inputs and to vary process material feed rates and carbonaceous material feed rates, to optimize the production of a synthetic gas mixture on-the-fly to react to changes in feedstock composition and changes in microbial digestion demands, to achieve maximal operational efficiency (noting that systems that must be operated at fixed output levels may at different times be producing either too little, or too much of the synthetic gas mixture).

Synthetic gas production based on partial oxidation techniques generate synthetic gas mixtures that include a CO₂ component, and CO₂ may not be required by the microbes, and in some cases may be detrimental to the downstream microbes, in which case additional effort/energy is required to remove the CO₂ fraction from the synthetic gas mixture, reducing the efficiency of the system. An important aspect of the concepts disclosed herein is a method of production that can produce synthetic gas mixtures low in or devoid of CO₂.

Conventional processes for producing synthetic gas mixtures are sensitive to contaminants in the feedstock. For example, organic feedstock often contain such high levels of sulfur that the sulfur must be removed prior to processing, because sulfur will poison the catalysts on which most commercial synthetic gas processes rely. Desulfurization involves additional process steps and expense. It would be desirable to produce synthetic gas mixtures from sulfur-containing feedstock without requiring pretreatment to remove the sulfur contaminant. An important aspect of the concepts disclosed herein is a method that can easily produce synthetic gas mixtures without the need for the removal of contaminants such as sulfur from the feedstock.

The plasma process herein can produce a tailored synthetic gas mixture required for the microbial digestion without the need for additional capital equipment or process steps. Plasma systems can vary the plasma power as well as using a plasma gas that is more conducive to the chemical process and to tailoring the synthetic gas mixture to meet the needs of the downstream digester. In the concepts disclosed herein, the plasma generated chemically reactive plasma can achieve a synthetic gas mixture having an exacting stoichiometric composition, such that the synthetic gas mixture produced can be tailored to the requirements of the microbial digestion/fermentation. With a plasma torch, more H₂ can be introduced through a methane or steam plasma, and more carbon can be introduced as a CO₂ plasma, as needed on-the-fly. Other reactants such as carbon black (or other carbonaceous materials), water, methane, oxygen, CO₂, etc. can be added into the reaction vessel to produce synthetic gas mixtures having a desired stoichiometric composition. The composition of the synthetic gas mixture can also be altered by varying the plasma power, and/or one or more of the reactants/process gases.

Synthetic gas mixtures have a long history as a chemical feedstock for the production of a wide variety of materials such as alcohols, aldehydes, acrylic acid, and ammonia through catalytic manufacturing processes. The concepts disclosed herein describe generating a tailored synthetic gas mixture for use as a feedstock for microbial digestion using a plasma system, where the plasma reactor can be controlled in real-time to produce a tailored gas composition to meet the needs of the downstream bio-reactor/digester. Significantly, changing the composition of the synthetic gas mixture can be used to improve the system efficiency, to temporarily encourage microbe growth over product production for digester repopulation after a system upset, and depending on the microbes being employed, changing the composition of the synthetic gas mixture can even result in changing the chemical product being produced in the bio-reactor. The latter aspect allows a commercial operation to adjust to market conditions on-the-fly; thereby producing the more lucrative product as market conditions change, without changes in the synthetic gas production equipment.

Present commercial syngas technology yields a product whose H₂:CO ratio varies from as high as 6:1 to as low as 3:2. There are some applications for synthetic gas mixtures in which excess H₂ is desired, but more frequently CO is the more useful component of a synthetic gas mixture, and thus, a lower ratio is more desirable. Conventional processes for production of synthetic gas mixtures that are capable of achieving low H₂:CO ratios typically do so by using a CO₂ recycle technology in which a product gas has a CO₂ impurity removed (CO₂ is formed as a byproduct in conventional synthetic gas production as a result of oxidation reactions in the reaction vessel). The recovered CO₂ is then re-injected into the reaction vessel, yielding a synthetic gas mixture having a low ratio. While this technique produces synthetic gas mixtures having lower ratios, it involves additional process steps and expense. The plasma reformation techniques disclosed herein can achieve a low H₂:CO ratio without the need to utilize a CO₂ recycle step in the process.

Impurities are also introduced into the resultant synthetic gas mixture in conventional processes as a byproduct of the reaction process. Conventional steam reforming introduces H₂O vapor and CO₂ that must be removed. Combustion-based reactions also introduce H₂O vapor and CO₂, thus diluting the synthetic gas mixture produced and can also introduce nitrogen oxide (NOₓ) emissions and soot, which are contaminants requiring removal. Again, removal of these contaminants involves additional process steps and expense. Further, the presence of CO₂ in the synthetic gas mixture may adversely affect pH level in the microbial environment to a degree that is detrimental to product production or microbial health. The plasma reformation techniques disclosed herein can produce synthetic gas mixtures efficiently without the need to provide for the removal of diluents, such as H₂O vapor and CO₂, or contaminants, such as NOₓ and soot.

Process parameters can be changed in conventional processes for the production of synthetic gas mixtures to enable the ratio of components to be varied, but only over a relatively narrow range. Large-scale changes in the component ratio require the additional steps of ratio enhancement and/or separation of overproduced components, representing added steps and expense. Furthermore, each non-plasma based process to produce synthetic gas mixtures has a characteristic range of component ratios that can be produced by that process. Before a synthetic gas mixture production facility is constructed, it is critical to know what the desired component ratio is, because the ratio desired would determine the process most suited to produce that ratio. Once the facility is constructed, adding ratio enhancement equipment to achieve different ratios is possible, but time consuming and expensive as well. Moreover, synthetic gas mixture production facilities are often part of a larger petrochemical production facility, and the ratio of the synthetic gas mixture required by such facilities can vary. The plasma reformation techniques disclosed herein can vary the synthetic gas mixture ratio over a relatively wide range without the use of costly ratio enhancement techniques, so that synthetic gas mixture production can be tailored to the varying needs of a site or based on economic conditions. If a selected product has more favorable economics, then it is desirable to be able to vary the composition of the synthetic gas mixture to achieve higher production of said product. The method disclosed herein enables synthetic gas mixtures having a specific composition (and specific ratio of components) to be produced simply by selectively introducing readily available reactants such as steam or CO₂, along with an organic feed or by changing the organic feed. For example, if higher H₂:CO ratios are desired, steam in the form of a plasma and/or feed reactant can be introduced. If lower H₂:CO ratios are desired, carbon dioxide in the form of a plasma and/or feed reactant can be introduced.

Finally, many non plasma based methods to produce synthetic gas mixtures rely on reaction vessels that operate under high pressure. Such vessels are often more costly to build and operate than vessels that operate at much lower pressures. Furthermore, reactants can only be introduced into such high-pressure reaction vessels at the elevated operating pressure. Finally, pressure reduction may be required to accommodate environmental conditions of the microbial digesters. The plasma reformation techniques disclosed herein can produce synthetic gas mixtures in a reaction vessel that operates at relatively low pressures, so it is not necessary to supply the feedstock at a high pressure and can more easily match required microbe environmental conditions.

Conversion of synthetic gas mixtures into other chemical products is typically performed under high pressure via catalysts. Examples include N₂ and H₂ conversion to ammonia which is later converted into fertilizers, CO and H₂ converted into methanol via the Fischer-Tropsch process, and so on. Recently, progress has been made in capturing microbes to perform the same conversion through aerobic or anaerobic digestion. Microbes have been discovered that preferentially produce ethanol, butanol, and other higher alcohols, or ethyl acetate based on specific conditions of H₂, CO, CO₂ and water present. Cyanobacteria are known to create triglyceride fatty acids, which can be used as a diesel fuel. Work is ongoing to genetically modify microbes and enzymes to directly produce hydrocarbons, and the microbes and enzymes might be used in a microbial digester/bio-reactor, as described herein. The concepts disclosed herein use plasma to generate a synthetic gas mixture that is introduced into a microbial digester/bio-reactor, where microbes convert the synthetic gas mixture into a desired material. The stoichiometric ratio of the synthetic gas mixture (along with selected additional chemical additives required by specific microbes) produced using plasma can be readily varied, so that the specific microbes can be varied, without requiring structural changes to the plasma portion of the system (only the operating parameters of the plasma portion of the system are changed).

### Details of Exemplary But Not Limiting Embodiments

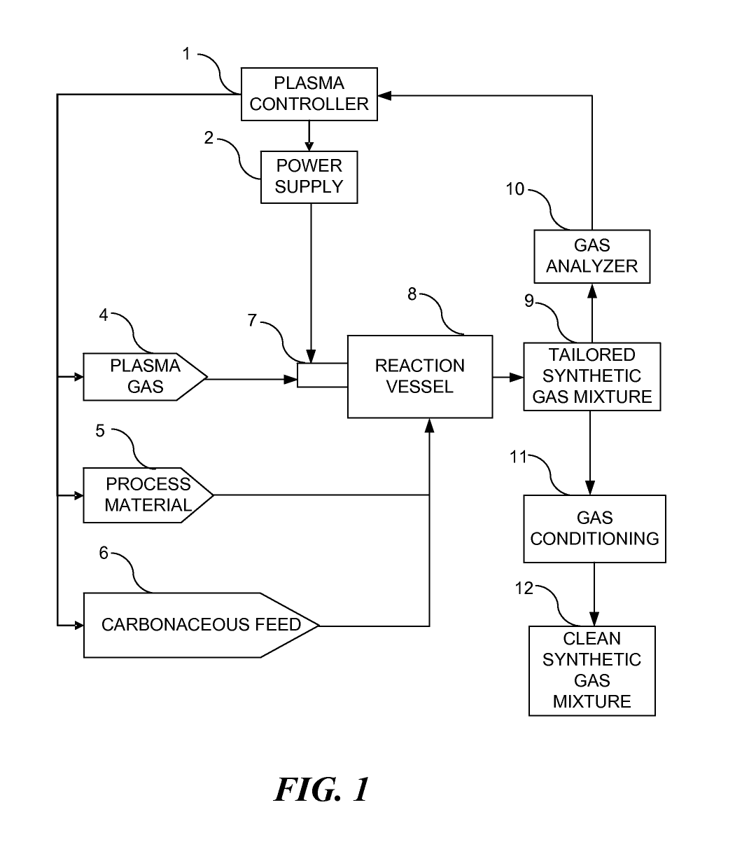

FIG. 1 is a functional block diagram illustrating an exemplary embodiment of the concepts disclosed herein, incorporating a plasma controller 1 (i.e., a programmable control device, such as a processor that executes machine instructions stored in a memory, or a hardware based control circuit), which controls a power supply 2, which provides electrical energy to plasma torch 7. Plasma controller 1 also controls a feed rate of plasma gas from a plasma gas supply 4 (such plasma gases being ionized by the plasma torch), and a feed rate of process material from a process material supply 5 (such process material being used for achieving a desired composition of the synthetic gas mixture), and a feed rate of carbonaceous material from a carbonaceous feed or material supply 6 (the carbonaceous material similarly being used for achieving the desired composition of the synthetic gas mixture). An operator starts the plasma gas and ignites plasma torch 7. Once the plasma torch is operating, the operator sets the feed rates for plasma gas 4, process material 5, and carbonaceous feed 6. The plasma gas, any process material, and any carbonaceous feed material are introduced into a reaction vessel 8, in which the energetic plasma reforms the materials to generate a tailored synthetic gas mixture 9, which can be collected from an outlet of the reaction vessel. A gas analyzer 10 determines a relative amount of each component of the tailored synthetic gas mixture (such as the relative amounts of H₂ and CO, and if desired, the relative amounts of any other residual components, such as NOₓ, SOₓ, and CO₂). Based on the required composition of the synthetic gas mixture (as indicated by baseline conditions determined for the downstream bio-reactor/digester), the operator or the controller can then adjust one or more of the plasma gas feed rate, the process material feed rate or the carbonaceous feed rate to obtain the correct composition of the tailored synthetic gas mixture. Note that the synthetic gas mixture may need to be conditioned by a gas conditioning component 11 before being introduced into a downstream bio-reactor/digester. Such a component can be used to adjust a temperature or pressure of the synthetic gas mixture, or to clean the synthetic gas mixture to remove any potential contaminants (such as halogens, NOₓ, SOₓ, ash, etc.). A clean synthetic gas mixture 12 is then ready to be introduced into a downstream bio-reactor/digester, so that microbes can convert the clean synthetic gas mixture to a different chemical product (or products).

Typical plasma gases include air, helium, argon, CO₂, O₂, and steam. CO₂, O₂, and steam can behave as chemically reactive species to produce the desired composition of the tailored synthetic gas mixture. For example, if methane is used as the carbonaceous material, a CO₂ plasma will create a synthetic gas mixture having a ratio of H₂:CO of about 1:1. Using a steam plasma with a methane feed would create a tailored synthetic gas mixture having a ratio of H₂:CO of about 3:1, which is a very different stoichiometric ratio. The desired stoichiometric ratio depends on the requirements of the microbial digester. The process materials can be any material that contributes to the production of the synthetic gas mixture, including but not limited to materials such as O₂, N₂, H₂, and water. The amount of process material that is provided to the reaction vessel can be varied across a wide range (including the option to provide no process material), depending on the size of the reaction vessel and the desired composition of the synthetic gas mixture. The carbonaceous feedstock can be any carbon source, including but not limited to, carbon black, coal, coke, methane, petroleum, biomass, etc., and the amount of carbonaceous feedstock introduced into the reaction vessel can also be varied across a wide range (including the option to provide no carbonaceous material), depending on the size of the reaction vessel and the desired composition of the tailored synthetic gas mixture.

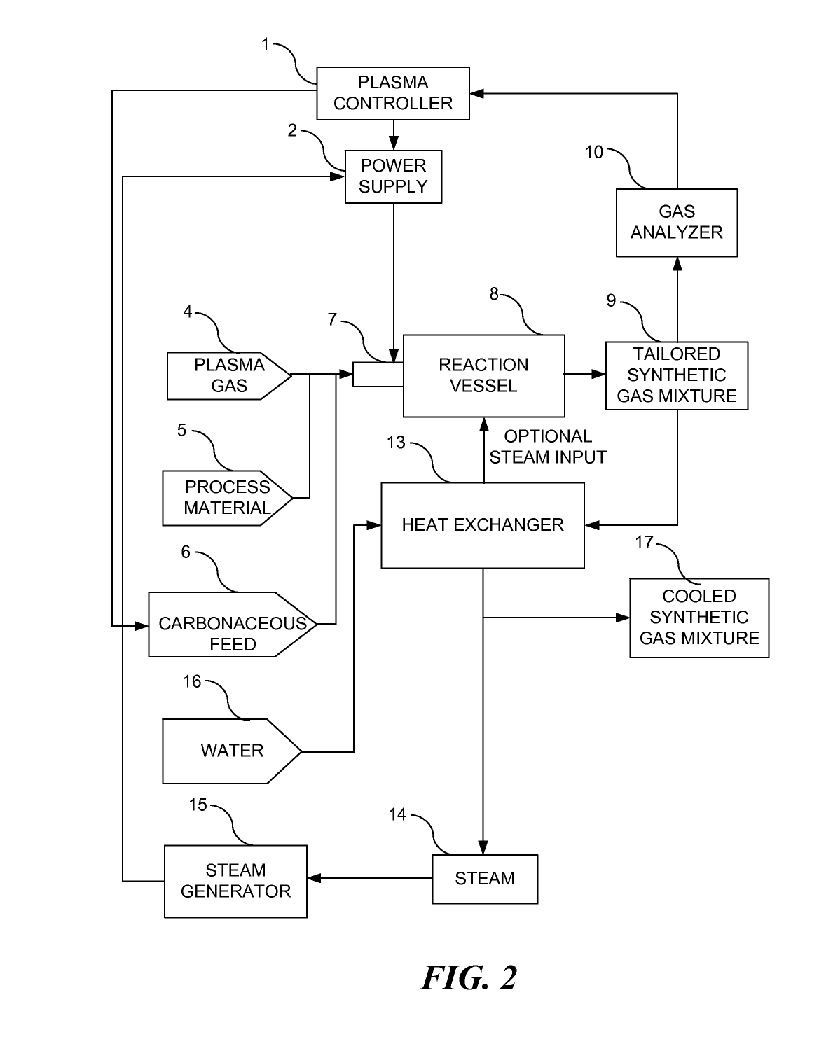

FIG. 2 is a functional block diagram illustrating an embodiment of the concepts disclosed herein that captures thermal energy from the synthetic gas mixture via a heat exchanger 13, in order to preheat the process materials and carbonaceous feed materials, or to produce electricity through a steam turbine, to provide electric power that can be used to energize the plasma torch (or to supply electrical power to some other system component). Plasma-based production of synthetic gas mixtures typically occurs at temperatures greater than about 900° C., especially if the target synthetic gas mixture contains CO and H₂. Reaction temperatures can be lower depending on the composition of the synthetic gas mixture desired. For example, lower temperatures can produce a synthetic gas mixture including relatively larger amounts of CO₂ and methane. Microbial digesters typically operate at ambient temperatures, or temperatures slightly above ambient. Therefore, the temperature of the tailored synthetic gas mixture generally is too high and requires cooling. Cooling tailored synthetic gas mixture 9 that is exiting the plasma reactor using heat exchanger 13 produces steam 14, which can run a steam generator 15 to produce electric power. If desired, part (or all) of the steam from heat exchanger 13 can be diverted to reaction vessel 8 for use as a process material. Gas analyzer 10 can provide data on the composition of the tailored synthetic gas mixture to plasma controller 1, and the plasma controller can use that data to determine if the quantity of steam being introduced into the reaction vessel should be increased or decreased, based on the desired composition of the synthetic gas mixture. Depending on the volume of synthetic gas mixture produced and the temperature difference between the synthetic gas mixture exiting the plasma reactor and the synthetic gas mixture temperature required by the microbial digester, the steam generator may provide enough power to energize the plasma torch. Water 16 is supplied to the heat exchanger to generate steam 14, and a cooled synthetic gas mixture 17 exits the heat exchanger.

In addition to generating onsite power through the thermal load of the exiting synthetic gas mixture, any process material 5 or carbonaceous feed 6 can optionally be preheated using thermal energy from the heat exchanger, to heat that material to a temperature closer to the reaction temperatures within the plasma reactor, which in turn, lowers the amount of thermal energy that needs to be provided by the heated plasma gas (thus reducing the electrical energy required to generated the heated plasma). Optimization of power production and pre-heating reaction materials depends on the application, and the artisan of ordinary skill will be able to readily determine whether it is more efficient to emphasize using the scavenged thermal energy for preheating, or electrical power production, or a combination of the two.

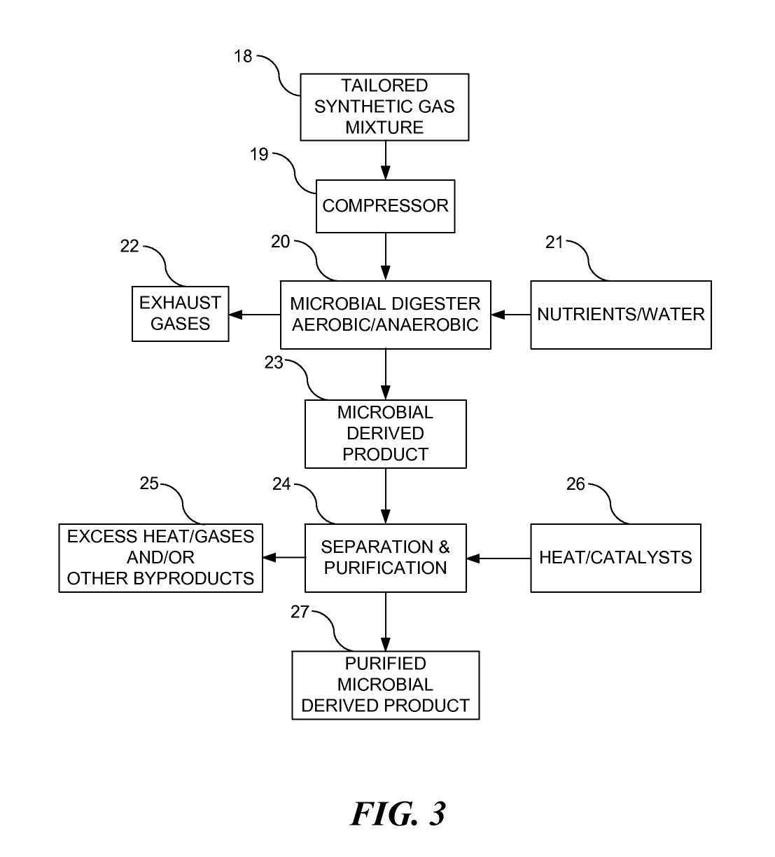

FIG. 3 is a functional block diagram that illustrates an embodiment of the concepts disclosed herein that introduces a tailored synthetic gas mixture 18 (appropriately cleaned, cooled, and/or treated, as discussed above) to a microbial digester 20 (optionally through a compressor 19), to produce a desired chemical product (or products, i.e., the targeted material) via microbial digestion or fermentation. The cooled tailored synthetic gas mixture may be compressed via a compressor 19 if the microbial digester requires a higher than ambient pressure for the tailored synthetic gas mixture (note that the plasma reactor generally operates at ambient pressure). If no increase in pressure is required, then only enough compression is employed to introduce the tailored synthetic gas mixture into microbial digester 20. The microbial digester contains the microbes that convert the tailored synthetic gas mixture into the targeted material. Microbial digester 20 may be a continuously stirred tank, a thin film substrate, a solid state substrate or any vessel containing a medium in which the microbes grow and ferment the tailored synthetic gas mixture into the desired product or ingest the tailored synthetic gas mixture and excrete the desired product. The microbial digestion can occur under aerobic or anaerobic conditions, depending on the microbes used, and on the conditions required for the microbes to produce the targeted material. The system injects any nutrients 21, additional water, or any medium required by the microbe to produce the targeted material. For example, certain microbes can produce ethanol from a tailored synthetic gas mixture that includes CO and H₂. The system injects the CO and H₂ tailored synthetic gas mixture into the microbial digester, where the microbes convert the tailored synthetic gas mixture into ethanol. Exiting the digester is a microbial derived product stream 23, which includes the desired product (such as ethanol), as well as byproducts such as off-gases 22. Depending on the composition of such off-gases 22, they can be captured and introduced into the plasma reactor to be used to generate more of the synthetic gas mixture. The targeted material may need to be separated from such byproducts, as indicated by a separation and purification process 24. For example, if the microbial digester produces ethanol to be used as a fuel, and the ethanol is mixed with water, the ethanol would need to be separated from the water fraction. The separation/purification process is a function of the intended use of the targeted material, and the desired purity. Heat or catalysts 26 may be required to separate the target material from the byproducts. The separation/purification process generates byproducts 25 (such as heat or off-gases) and a purified product 27. For example, ethanol distillation uses heat and sodium hydroxide to precipitate out any organic acids and the sodium organic acid salt, which represent byproducts 25 for such a purification step.

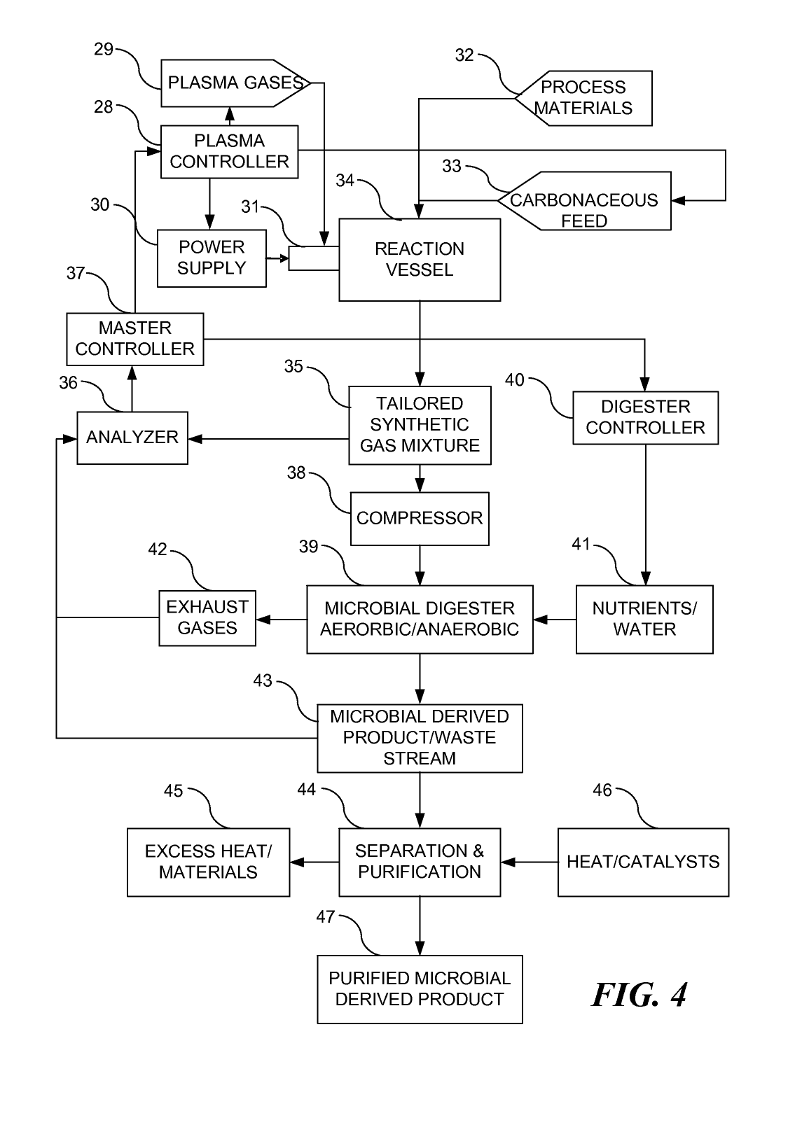

FIG. 4 is a functional block diagram illustrating an embodiment of the concepts disclosed herein that uses a feedback loop to increase system efficiency, by collecting system data that is used to change the operating parameters of the plasma torch, so that the quality and quantity of the tailored synthetic gas mixture produced in the plasma reactor is matched to the needs of the microbial digester/bio-reactor. Fine tuning the composition of the tailored synthetic gas mixture enables the maximum yield of microbial product to be achieved, with the least amount of the tailored synthetic gas mixture, or such fine tuning can be used to induce the microbes to produce a different product by changing the composition of the synthetic gas mixture, or such fine tuning can be used to induce accelerated microbial growth over product production (typically by providing excess CO) when desired (for example, as may be needed for tank repopulation after a system upset event). A plasma controller 28 controls the input of plasma gases 29 and the electrical power supplied by a power supply 30 to a plasma torch 31, as well as the feed rates of process materials 32 and a carbonaceous feed 33 into a reaction vessel 34, in which a synthetic gas mixture 35 is produced. An analyzer 36 measures the composition of the tailored synthetic gas mixture and sends the data to a master controller 37. (It should be understood that plasma controller 28 and master controller 37 can be implemented using a single controller if desired; further, it should be understood that any control function required by the system can be implemented using a single master controller or individual controllers that are each responsible for one or more control functions). Based on the tailored synthetic gas mixture composition data, master controller 37 directs plasma controller 28 to make any changes in the operation of plasma torch 31, or the feed rates for process materials 32 feed or carbonaceous feed 33. This feedback process continues until a desired composition of the tailored synthetic gas mixture is obtained.

Referring again to FIG. 4, if a downstream microbial digester 39 is a pressurized vessel, tailored synthetic gas mixture 35 is pressurized via a compressor 38 to the appropriate pressure required for operation of the microbial digester. Tailored synthetic gas mixture 35 is introduced into microbial digester 39 for microbial digestion/fermentation. A digester controller 40 controls the introduction of nutrients/water 41 (i.e., nutrients in water and/or other media) into microbial digester 39, to facilitate microbial growth, and to control characteristics of the microbial derived product. Microbial digester 39 releases exhaust gases 42 and a raw microbial derived product/waste stream 43 (i.e., the output from the microbial digester includes the microbial derived product and waste stream or byproducts from which the desired product will be separated and purified, generally as discussed above). Analyzer 36 monitors exhaust gases 42 and microbial derived product/waste stream 43 from microbial digester 39 for their respective compositions, including the composition of the exhaust gases, the type and amount of the product exiting microbial digester 39, and the environmental conditions in the digester. The analyzer sends the data to master controller 37, which instructs plasma controller 28 and digester controller 40 to alter conditions in the plasma reactor and the microbial digester as required, based on the process feedback. If no changes are required, no action is taken. Microbial derived raw product/waste stream 43 enters a separation and purification stage 44, which may use heat and catalysts 46 to purify the targeted product. Any excess heat or byproduct materials 45 from the separation and purification stage can be re-introduced into the microbial digester as nutrients or as process materials for the production of the tailored synthetic gas mixture, if such introduction would increase system efficiency. A purified product 47 is collected from separation and purification stage 44.

If the microbes housed in microbial digester 39 can produce different products based on the composition of the tailored synthetic gas mixture, the operator can use master controller 37 to change conditions in the plasma reactor and microbial digester as required, to change the targeted product 47 produced in the microbial digester. For example, some microbes will selectively produce ethanol or acetate based on the tailored synthetic gas mixture composition and microbial digester conditions. Master controller 37 uses stored information to cause plasma controller 28 to change the plasma process conditions as required to generate a synthetic gas mixture 35 having a different composition. Plasma controller 28 can change one or more of the following parameters to change the composition of the tailored synthetic gas mixture: plasma gases 29, power provided by power supply 30, process materials feed 32, and carbonaceous feed 33. Note that such changes can be based not only on relative quantities of the plasma gas, the process material, and/or the carbonaceous feed material, but also the specific ones of such material (that is, a different plasma gas can be used, a different process material can be used, or a different carbonaceous feedstock can be used). Analyzer 36 will monitor a composition of tailored synthetic gas mixture 35 and provide data about the composition of the tailored synthetic gas mixture to master controller 37, which will then instruct plasma controller 28 to alter its conditions to change the composition of tailored synthetic gas mixture 35, as required. This process will continue until the composition of tailored synthetic gas mixture 35 being produced corresponds to a desired composition and condition. Analyzer 36 monitors exhaust gases 42 and raw microbial derived product/waste stream 43 from microbial digester 39 to determine their respective compositions, including the composition of the exhaust gases, the type and amount of the product exiting microbial digester 39 and the environmental conditions of the microbial digester. The analyzer sends the data to master controller 37, which instructs plasma controller 28 and digester controller 40 to alter the conditions in the plasma reactor and microbial digester as required based on the process feedback. If no changes are required, no action is taken.

Methanogenic bacteria can produce methane from a synthetic gas including CO₂ and H₂. Details of this conversion are provided in Equation (1), as follows.

CO₂+4H₂→CH₄+2H₂O (1)

Methane is a very valuable commodity known as natural gas. Therefore, low value feedstocks, such as waste biomass material (for example, straw or discarded municipal solid waste) provides a carbonaceous feedstock that can be converted into a tailored synthetic gas mixture, which microbes will convert into methane. In an exemplary but not limiting embodiment, straw is used as the carbonaceous feed source, straw being a largely cellulosic feedstock. Cellulosic feedstocks produce synthetic gas mixtures of H₂:CO on the order of about 1:1. Through a water gas shift reaction given in Equation (2), the CO from the cellulosic synthetic gas mixture can be converted to CO₂ and H₂ as follows.

CO+H₂O→CO₂+H₂ (2)

Using the water gas shift reaction will yield a H₂:CO₂ ratio of about 2:1. This ratio is below the ideal ratio H₂:CO₂ ratio of 4:1, but higher value carbonaceous materials would be required to achieve the 4:1 ratio. Therefore, one would expect an excess amount of CO₂ to emerge from the microbial digester (since the synthetic gas mixture from the relatively lower value cellulosic feedstock generates a synthetic gas mixture including about 2× the quantity of CO₂ required by the digester).

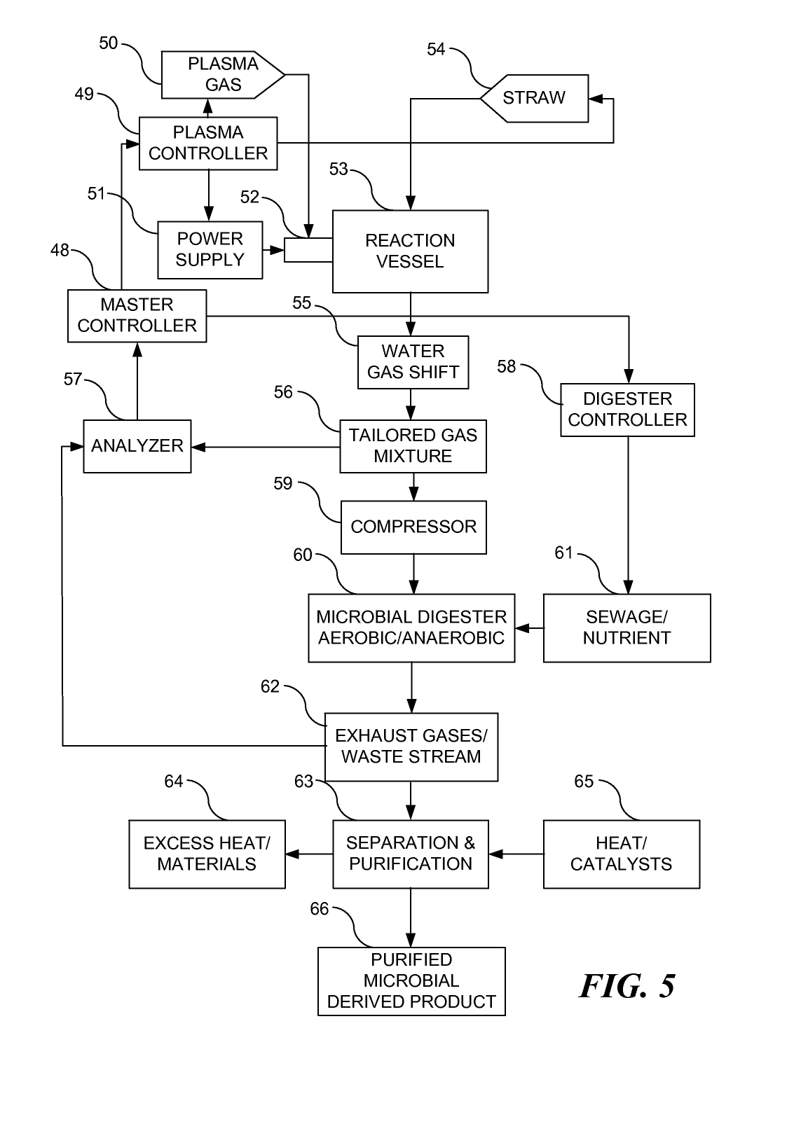

FIG. 5 is functional block diagram illustrating an exemplary embodiment of the concepts disclosed herein in which plasma torch is used to generate the desired synthetic gas mixture (i.e., a synthetic gas mixture whose components, and the stoichiometric ratio of those components, have been matched to the needs of the digester), and microbial digestion of the synthetic gas mixture is used to produce methane (CH₄). In this embodiment, straw 54 is used as the carbonaceous feed for production of a tailored synthetic gas mixture 56, and sewage 61 is used as a nutrient for the microbial digestion (sewage is typically digested into methane). It should be noted that other relatively low value carbonaceous feedstocks could be similarly employed, such as sawdust, wood waste, crop residues, and other types of biomass. The operator instructs a master controller 48 that methane is the desired microbial product and that straw 54 is the carbonaceous feedstock. A plasma controller 49 selects steam as a plasma gas 50, since steam reforming of free carbon will produce a tailored synthetic gas mixture having relatively larger amounts of H₂, which is favorable for the microbial conversion of that tailored synthetic gas mixture to methane. Plasma controller 49 sets power produced by a power supply 51 as required for generation of steam plasma gas 50 using a plasma torch 52 and controls the feed rate of straw 54 to produce tailored synthetic gas mixture 56 having the desired stoichiometric ratio. The straw and plasma gases are introduced into a reaction vessel 53 where the tailored synthetic gas mixture is produced. The tailored synthetic gas mixture goes through a gas conditioning process in the form of a water gas shift reaction 55 to increase a quantity of CO₂ in tailored synthetic gas mixture 56. An analyzer 57 monitors a composition of tailored synthetic gas mixture 56, and feeds the data to master controller 48. If the composition of the tailored synthetic gas mixture needs altering, the master controller instructs the plasma controller to alter the plasma conditions (plasma gas composition, plasma gas feed rate, plasma power levels, straw feed rate, etc.) to produce a tailored synthetic gas mixture having the desired gaseous components in the desired quantity and quality to support microbial conversion of that tailored synthetic gas mixture to methane.

Water shifted tailored synthetic gas mixture 56 is introduced into a microbial digester 60 with the aid of a compressor 59. A digester controller 58 introduces sewage 61 into a microbial digester 60 as the nutrient. A raw product comprising exhaust gases and waste stream 62 includes methane and any excess CO₂ or H₂. Analyzer 57 monitors the composition of the raw product and sends the data to master controller 48. If master controller 48 detects any H₂ in the exhaust gases, then the tailored synthetic gas mixture is providing more H₂ than the microbes can digest. In response, master controller 48 directs plasma controller 49 to lower the steam composition of plasma gas 50 to generate less H₂, and/or to lower the feed rate of straw 54 to lower the amount of the tailored synthetic gas mixture being produced. If the exhaust gas has more CO₂ than the ½ stoichiometry expected, then the microbes need more H₂, and master controller 48 directs plasma controller 49 to increase the steam composition of the plasma gas to increase hydrogen production.

Raw product (i.e., exhaust gases/waste) stream 62 from microbial digester 60 can also include waste biomass, if the sewage is not completely digested by the microbes. Any excess heat/materials 64, such as a waste byproduct can be re-introduced into the plasma reactor along with straw 54 as a biomass source for production of the desired tailored synthetic gas mixture. Raw product stream 62 is introduced into a separation and purification component 63, where any excess CO₂ is separated from the methane via absorption or the use of heat or catalysts 65, such as calcium hydroxide. In this example, a purified microbial derived product 66 produced by the separation and purification process is methane, which can be pumped into a natural gas pipeline or used for other applications, such as on-site power generation.

A specific microbe, Clostridium ljungdahlii, will preferentially produce ethanol or acetate from a synthetic gas mixture including H₂ and CO, based on the pH of the digestion medium. If the pH is between 4.0 and 4.5, the microbe produces ethanol. If the pH is between 5.0 and 7.0, the microbe produces acetate. Therefore, an operator can produce ethanol preferentially, or acetate preferentially, simply by altering the pH of the microbial growth medium. This ability gives the operator the advantage of selectively producing either product, based on market conditions. If ethanol production provides more profit than acetate production, or vice versa, the product being produced can easily be changed without requiring capital investment. The pH in the digester can be readily manipulated using hydrochloric acid or sodium hydroxide to alter the pH. However, the production of acetate or ethanol can also be manipulated by changing the stoichiometric ratio of the tailored synthetic gas mixture introduced into the microbial digester, eliminating the need and expense of handling corrosive substances. Dissolved CO₂ will lower the pH of an aqueous solution, and therefore, having a higher or lower partial pressure of CO₂ in the tailored synthetic gas mixture will lower or raise the pH, respectively.

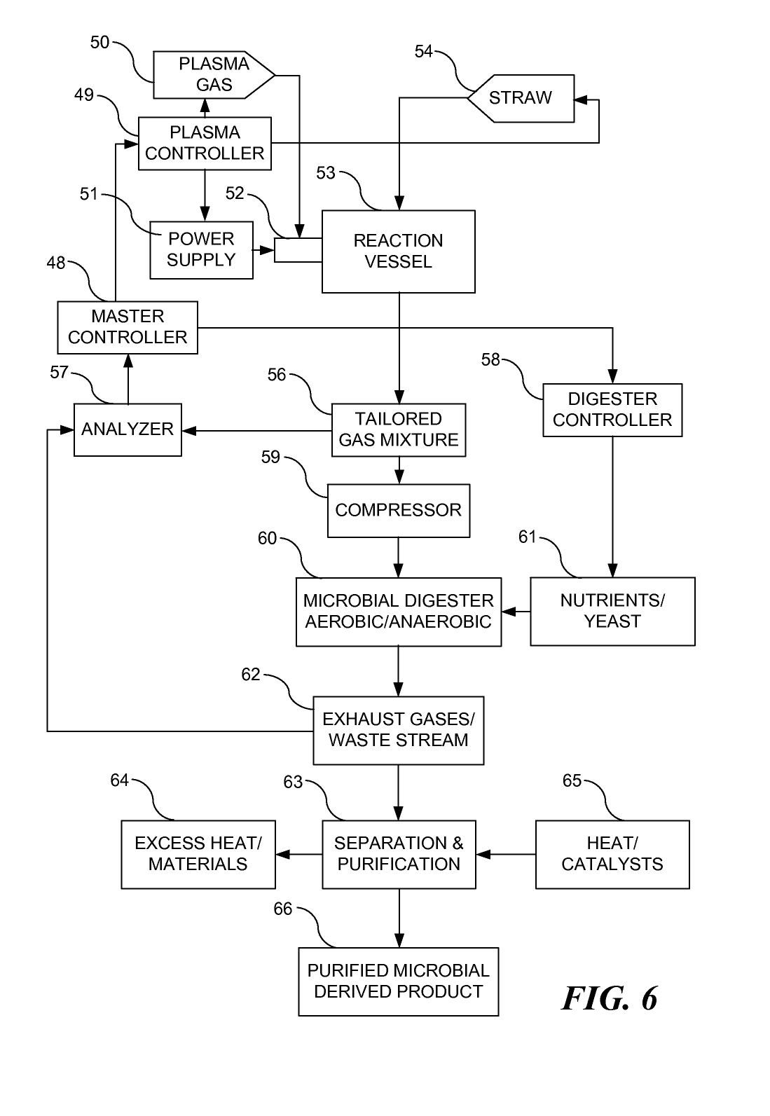

FIG. 6 is a functional block diagram of an exemplary embodiment in which a plasma torch is used to produce a synthetic gas mixture tailored to the needs of a downstream microbial digester, in which the synthetic gas mixture is selectively converted to ethanol or acetate, based on the composition of the synthetic gas mixture introduced into the digester. Preferably, this embodiment converts a low value carbonaceous feedstock to produce higher value products (i.e., ethanol or acetate). Thus, in a particularly preferred but not limiting embodiment, straw (or some other inexpensive and readily available biomass) is used as the carbonaceous feedstock to produce the synthetic gas mixture tailored to the needs of the downstream microbial digester. Where acetate production is desired, the operator selects acetate as the product via master controller 48, and the carbonaceous feed as straw 54. Plasma controller 49 sets plasma gas 50 to employ steam so as to minimize the production of CO₂, and thus, to maintain a pH between 5.0 and 7.0 in the microbial digester. Plasma controller 49 sets the power (at power supply 51) required for using plasma torch 52 to generate the steam plasma and sets the feed rate of straw 54 to produce tailored synthetic gas mixture 56 in a desired quantity and quality. Straw 54 and plasma gas 50 are introduced into reaction vessel 53, where tailored synthetic gas mixture 56 is produced. Analyzer 57 monitors the tailored synthetic gas composition and sends the data to master controller 48. If the composition of the tailored synthetic gas mixture needs to be altered, the master controller instructs plasma controller 49 to alter the plasma gas composition, or the straw feed rate, or any other condition that will modify the composition of the tailored synthetic gas mixture to the needs of the downstream microbial digester. Tailored synthetic gas mixture 56 is introduced into microbial digester 60 with the aid of compressor 59. Digester controller 58 introduces any nutrients/yeast 61 (or additional media) that are required into the microbial digester. Microbial digester 60 releases any exhaust gases/waste 62 as a raw product stream, which includes the acetate and byproducts. Analyzer 57 monitors the exhaust gases. If too much H₂ and CO are exiting microbial digester 60 in the raw product stream, then too much tailored synthetic gas mixture 56 is entering microbial digester 60. The analyzer sends the data to master controller 48, and the master controller instructs plasma controller 49 to reduce the feed rate of straw 54 in order to produce less of tailored synthetic gas mixture 56. Plasma controller 49 adjusts the power from power supply 51 to compensate for a lower feed rate of straw 54. The raw product stream comprising exhaust gases/waste stream 62 with acetate enters separation and purification component 63, which can separate the acetate from byproducts by adding heat/catalysts 65 (e.g., sodium hydroxide) to precipitate sodium acetate as purified microbial derived product 66. Any excess heat or waste material 64 can be re-introduced into the plasma reactor with straw 54 feed to generate more of tailored synthetic gas mixture 56.

If the market price of ethanol increases, the operator can select the targeted product using master controller 48 to be ethanol. The master controller then instructs plasma controller 49 to add more CO₂ to tailored synthetic gas mixture 56, to change the composition of the tailored synthetic gas mixture in a manner that will change the product produced in the microbial digester. Master controller 48 instructs digester controller 58 to select appropriate nutrients for ethanol production. Plasma controller 49 adds more O₂ and CO₂ to plasma gas 50 to get a higher partial pressure of CO₂ in tailored synthetic gas mixture 56. Analyzer 57 monitors the composition of tailored synthetic gas mixture 56 and the pH of the raw product comprising exhaust gases/waste stream 62 and sends the data to master controller 48. Once the pH is lower than 4.5, the master controller instructs plasma controller 49 that it is producing tailored synthetic gas mixture 56 having the correct composition. Plasma controller 49 now has established the correct plasma conditions for producing ethanol. The raw product stream exiting microbial digester 60 enters separation and purification component 63, which can separate the ethanol using distillation. Now the system can change between ethanol and acetate production using the same feedstock for tailored synthetic gas production and nutrients microbial growth on-the-fly, e.g., in response to market conditions, yielding a much more lucrative commercial operation.

Another aspect of the concepts disclosed herein is the use of a plasma reactor to convert CO₂ emissions to ethanol. This technique can simultaneously reduce a significant environmental problem, CO₂ emissions, and use generated ethanol fuels to solve another problem, foreign oil dependence. Empirical studies indicate such a technique can use CO₂ emissions for the production of ethanol, resulting in a “Well to Wheels” CO₂ reduction of 50% over equivalent gasoline use. The technique is robust, scalable, and unobtrusive to existing CO₂ emitting facilities and produces high volumes of ethanol output.

The process uniquely combines chemical and biological technologies to consume CO₂ and other hydrocarbons as feedstocks for the production of ethanol. The process creates a clean and tailored synthetic gas mixture, which is then fed to a product specific microorganism that ingests the tailored synthetic gas mixture and rapidly excretes ethanol (as discussed above, composition of the tailored synthetic gas is controlled to meet the needs of the microbial digester, preferably in real-time). Such an ethanol plant is unobtrusive to existing CO₂ emitting facilities, requiring no special conditioning of the captured CO₂. The plants can be successfully co-located with almost any significant CO₂ source, with either diluted or concentrated CO₂ emissions. Some examples of such CO₂ sources are natural gas processing plants, coal-fired power plants, cement kilns, petroleum processing hydrogen plants and corn ethanol plants. Generally as discussed above, this CO₂ emission based process uses a plasma torch and thermal reaction chamber to gasify carbon materials along with CO₂, to produce a tailored synthetic gas mixture rich in carbon monoxide (CO) and hydrogen (H₂). The tailored synthetic gas mixture is then converted to ethanol using microbial digesters/bio-reactors. Significantly, this conversion of the tailored synthetic gas mixture to ethanol requires only a single step at low temperature and low pressure. The biological sub-system is robust, scalable and controllable to a level of zero air emissions.

A basic mass and energy balance evaluation of the overall process has been completed using actual operating data from sub-system vendors. The following conceptual design is of a proposed plasma/digester ethanol plant co-located with an existing 400 million SCF per day natural gas processing plant removing 6% CO₂ (55,000 kg/hr) using a standard amine separation system.

The feedstock combination is CO₂, methane, oxygen and Powder River Basin (PRB) coal (exemplary—not limiting, since other types of coal can instead be used). Other forms of carbon can be used depending on plant location; and the other carbon types can include higher value coal, petroleum coke, and even coal fines. The other feedstock components are simply adjusted to achieve complete CO₂ “destruction” and the desired tailored synthetic gas mixture composition for optimal ethanol production. The hydrogen component required in the tailored synthetic gas is obtained from the feedstock. There is no need for additional water to be introduced later in the ethanol production process. Plant electrical power is produced by a heat recovery/steam turbine sub-system from the heated tailored synthetic gas mixture, and onsite, natural gas-fired turbines, which keeps the plant “off grid.” This approach helps the overall process in two ways: power plant emissions are reduced by the avoidance of transmission line losses from a remote power plant; and, the onsite power plant's exhaust heat can be captured to aid in ethanol distillation. Feedstock gases are pre-heated using residual waste heat from the tailored synthetic gas mixture exiting the plasma reactor, before the feedstock gases enter the plasma reactor, to reduce the energy required to maintain the reactor temperature, thereby increasing the overall efficiency of the tailored synthetic gas mixture production process. The tailored synthetic gas mixture is then cooled and delivered to the microorganism located in the bio-reactor. The living microorganisms reside in standard fermentation reactors, where they ingest the tailored synthetic gas mixture and excrete ethanol in a rapid, product specific single step. A dilute ethanol/water mixture is continuously extracted, filtered, and processed to recover anhydrous ethanol in a standard molecular sieve distillation system. All CO₂ emitted in the distillation process is captured, ˜39 tonnes per hour, and added to the feedstock CO₂ from the natural gas processing plant, ˜55 tonnes per hour. The other feedstock material (O₂, CH₄, & PRB coal) is adjusted accordingly, to maintain the targeted composition of the tailored synthetic gas mixture.

The original consumed CO₂ feedstock of 55,440 kg/hr is offset by 25,100 kg/hr of CO₂ emitted by the production of the plant's electrical power needs, yet the result is still a net reduction of 30,340 kg/hr of CO₂. By also processing the 39,750 kg/hr of CO₂ captured from distillation, this plasma/digester ethanol plant consumes 3.8 times more CO₂ than it emits—making the entire process significantly CO₂ negative.

CO₂ Consumed in Production: 593,000 tons per year (including material transportation)

CO₂ Emitted when Combusted: 1,579,000 tons per year (blended as E10 in vehicles)

Total Well to Wheels CO₂ Emissions: 986,000 tons per year

The plasma reactors and ethanol bio-reactors operate continuously, eliminating the limitations and inefficiencies of current “batch processing” methods. Products of partial oxidation (CO & H₂) from the hydrocarbon feedstock used in the plasma reactor to help crack the CO₂ are not off gassed—they become part of the feedstocks for the microorganisms that create the ethanol. There is a near zero waste of carbon and energy. Water is not consumed in the fuel production process.

The process is “closed loop” and product specific, with no unwanted byproducts to mitigate, such as byproduct found in other alternative fuel production, which can include DDG, glycerin, chemical byproducts, effluents, etc. The process does not require design changes in existing CO₂ emitters' processes; it truly is “bolt on” technology.

The CO₂ feedstock is readily available in all geographical areas and climates. Most CO₂ emitting sources are located near major population centers where the resulting fuel is needed, thereby reducing fuel transportation costs and transportation related emissions. The process does not have direct or indirect land-use change issues common to crop-based fuel processes. A 100 million gallon plasma/digester ethanol plant would occupy just 20 acres—12,000 times less land than an equivalent corn ethanol plant and 4,000 times less than projected switch grass ethanol plants.

Note that ethanol is a potential feedstock for production of ethylene, which can be simply processed to produce polyethylene or plastic. The plasma/digester ethanol process can be used to consume CO₂ and produce ethanol as a feedstock for the manufacture of plastic, further displacing petroleum use and creating a CO₂ sink. Such an embodiment represents a 100% CO₂ reduction, since the CO₂ is permanently locked in the plastics matrix.

The 400 million SCF per day natural gas processing plant used in the model above actually exists 50 miles outside of Denver, Colo. and could be an excellent candidate for the first plant site. The 276 million gallons of ethanol produced at this single plasma/digester ethanol plant would be equal to 13% of the annual gasoline consumption for the entire state of Colorado.

There are currently 530 natural gas processing plants in the U.S. releasing enough CO₂ into the atmosphere to produce 30 billion gallons of ethanol per year via the plasma/digester process. Thirty billion gallons of ethanol would displace roughly 14% of the gasoline consumed in the U.S.

The CO₂ emitted from a single, 100 million gallon corn ethanol plant could be used to create another 162 million gallons of ethanol annually via a co-located plasma/digester ethanol plant. Ethanol production from corn could easily be doubled without requiring another acre of land to be farmed.

Cement kilns are the largest industrial emitters of CO₂. The annual U.S. production of cement is 100 million tonnes per year, with an emission of 120 million tonnes of CO₂ annually. Kilns are well distributed throughout the U.S. population centers, and a single, 1 million tonne per year cement kiln could co-produce 638 million gallons of ethanol using the plasma/digester process. The total market potential using CO₂ from cement kilns is 64 billion gallons of ethanol annually—enough to displace roughly 30% of the current gasoline consumed in the U.S.

It should also be noted that a substantial portion of the energy and cost associated with carbon capture and sequestration is in the sequestration step—to compress and transport the captured CO₂. This step is not necessary for the plasma/digester process. The captured CO₂ can be processed as it is presented in both temperature and pressure. There is no need to cool the captured CO₂ or raise its pressure as it enters the plasma reactor.

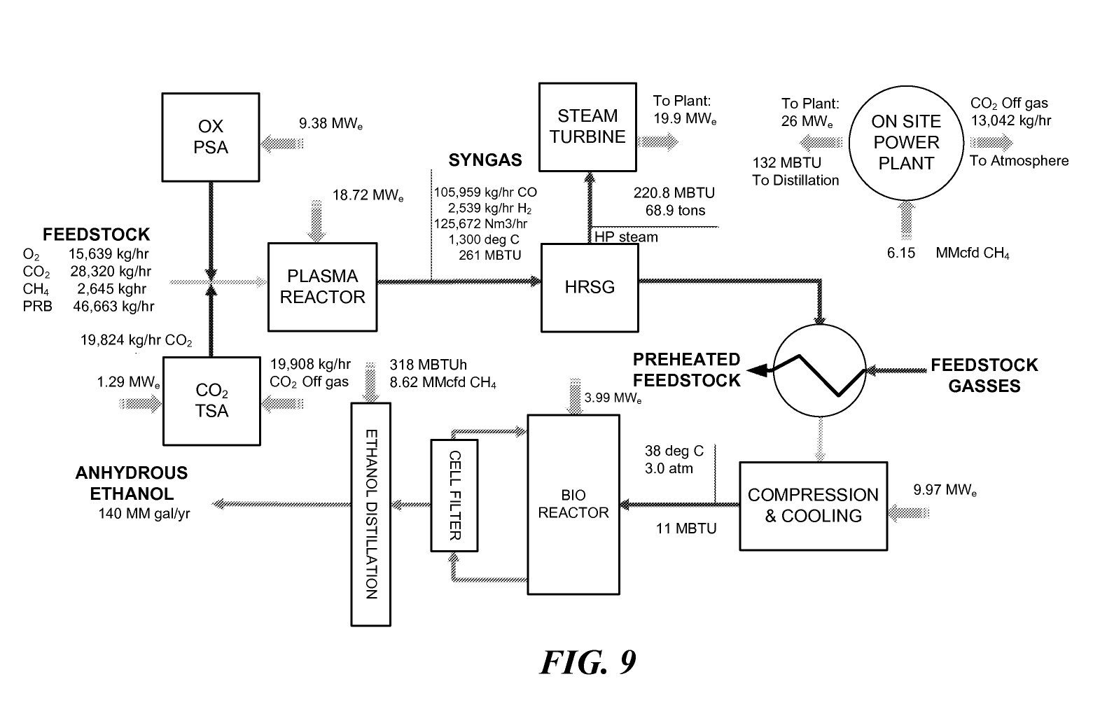

FIG. 9 is an exemplary functional block diagram and mass energy balance for an embodiment that simultaneously reduces CO₂ emissions from a CO₂ generating facility and produces ethanol, generally as discussed above. This Figure is self-explanatory and is based on the above description.

In the above description, reference has been made to the use of straw or sewage as possible system inputs. It should be recognized that such materials are intended to be exemplary of relatively low value materials that can be used as system feedstocks to produce relatively higher value products. It should be understood that other types of relatively low value feedstocks can be similarly employed. For example, straw simply represents one relatively low value biomass, but other types of biomass can be similarly employed.

With respect to the claims that follow, it should be understood that the term plasma reactor refers to a combination of a plasma torch and a reaction vessel. It should be recognized that manipulation of the plasma reactor (i.e., changing conditions in a plasma reactor) can be accomplished by changing operating parameters of the plasma torch, and/or by changing conditions in the reaction vessel (for example, inputs of reactants into the reaction vessel that are introduced into the reaction vessel independently of the plasma torch).

Finally, it should be noted that there is significant potential for engineered microorganisms to produce pharmacologically valuable compounds. The concepts disclosed herein can be used to provide a tailored synthetic gas mixture (using a plasma reactor generally as discussed above) to a bioreactor including a naturally occurring or engineered microorganism, which converts the tailored synthetic gas mixture into one or more pharmacologically valuable compounds. The pharmacologically valuable compound produced in the bioreactor may itself be a pharmacologically active compound, or may be a precursor used to produce a pharmacologically active compound. In a related exemplary embodiment, the tailored synthetic gas mixture can be converted to one or more amino acids in the bioreactor. The amino acids thus produced can be used to produce pharmaceuticals, as well as other chemical products.

Although the concepts disclosed herein have been described in connection with the preferred form of practicing them and modifications thereto, those of ordinary skill in the art will understand that many other modifications can be made thereto within the scope of the claims that follow. Accordingly, it is not intended that the scope of these concepts in any way be limited by the above description, but instead be determined entirely by reference to the claims that follow.

The invention in which an exclusive right is claimed is defined by the following:

1. A method for converting a carbonaceous feedstock into a chemical product having a higher value than the carbonaceous feedstock, comprising:
(a) introducing the carbonaceous feedstock into a plasma reactor that includes a plasma torch under conditions controlled to generate a synthetic gas mixture having a specific composition;
(b) introducing a quantity of the synthetic gas mixture into a microbial bioreactor configured to convert the synthetic gas mixture into the chemical product;
(c) varying the specific composition of the synthetic gas mixture introduced into the microbial bioreactor based on changing needs of the microbial bioreactor, where at least in part, the conditions in the plasma reactor are controlled to vary the specific composition of the synthetic gas mixture; and
(d) collecting the chemical product produced by the microbial bioreactor.

2. The method of claim 1, further comprising monitoring conditions in the microbial bioreactor to determine if the quantity of the synthetic gas mixture being introduced is sufficient to maximize a quantity of the chemical product being produced in the microbial bioreactor, and if not, then increasing the quantity of the synthetic gas mixture introduced into the microbial bioreactor.

3. The method of claim 1, further comprising monitoring conditions in the microbial bioreactor to determine if the quantity of the synthetic gas mixture being produced is in excess of an optimal quantity of the synthetic gas mixture required by the microbial bioreactor, and if so, then decreasing the quantity of the synthetic gas mixture introduced into the microbial bioreactor.

4. The method of claim 1, further comprising monitoring conditions in the microbial bioreactor to determine an optimal quantity of the synthetic gas mixture required by the microbial bioreactor for maximizing production of the chemical product, and then changing conditions in the plasma reactor as required to provide the optimal quantity of the synthetic gas mixture introduced into the microbial bioreactor.

5. The method of claim 1, further comprising monitoring conditions in the microbial bioreactor to determine an optimal stoichiometric ratio of the synthetic gas mixture required by the microbial bioreactor for maximizing production of the chemical product, and then changing the conditions in the plasma reactor as required to provide the optimal stoichiometric ratio.

6. The method of claim 1, further comprising monitoring conditions in the microbial bioreactor to determine an optimal quantity and composition of a synthetic gas mixture required by the microbial bioreactor for maximizing production of the chemical product, and then changing the conditions in the plasma reactor as required so that a composition and quantity of the synthetic gas mixture produced by the plasma reactor is tailored to provide requirements of the microbial bioreactor.

7. The method of claim 1, further comprising cooling the synthetic gas mixture exiting the plasma reactor to a temperature required in the microbial bioreactor, wherein cooling the synthetic gas mixture generates steam that is used to increase an efficiency of the method.

8. The method of claim 1, further comprising:
(a) analyzing the synthetic gas mixture exiting the plasma reactor;
(b) comparing a quantity and quality of the synthetic gas mixture produced by the plasma reactor to a quantity and quality of the synthetic gas mixture required by the microbial bioreactor to achieve a desired output of the chemical product; and
(c) modifying conditions in the plasma reactor so the quantity and quality of the synthetic gas mixture produced by the plasma reactor corresponds to the quantity and quality of the synthetic gas mixture required by the microbial bioreactor.

9. The method of claim 1, wherein the quantity and a quality of the synthetic gas mixture required by the microbial bioreactor is determined by at least one of the following:
(a) monitoring conditions in the microbial bioreactor;
(b) monitoring the chemical product produced by the microbial bioreactor; and
(c) monitoring an effluent exiting the microbial bioreactor that is not the chemical product.

10. The method of claim 1, wherein the carbonaceous feedstock is a biomass.

11. The method of claim 1, further comprising adding sewage to the microbial bioreactor as a nutrient.

12. The method of claim 1, wherein the chemical product comprises at least one product selected from the group of products consisting of:
(a) ethanol;
(b) butanol;
(c) higher alcohols;
(d) acetate;
(e) a pharmacological compound;
(f) a pharmacologically active compound; and
(g) an amino acid.

13. The method of claim 1, further comprising modifying conditions in the plasma reactor so that the quantity and a quality of the synthetic gas mixture produced by the plasma reactor and introduced into the microbial bioreactor results in production of a different chemical product.

14. The method of claim 13, wherein the chemical product comprises ethanol and the different chemical product comprises acetate.

15. The method of claim 1, further comprising the step of introducing an engineered microorganism into the microbial bioreactor, the engineered microorganism having been designed to produce a specific chemical product.

16. A system for converting a carbonaceous feedstock and a process material into a chemical product having a higher value than the carbonaceous feedstock, comprising:
(a) a plasma reactor configured to generate a synthetic gas, wherein the plasma reactor is controlled to generate a synthetic gas mixture having a specific composition;
(b) a microbial digester configured to convert the synthetic gas mixture into the chemical product; and
(c) means for monitoring one or more conditions in the microbial digester to provide feedback for controlling the plasma reactor so that the specific composition of the synthetic gas mixture is appropriate for converting the synthetic gas mixture into the chemical product.

17. The system of claim 16, further comprising a controller configured to control the plasma reactor to tailor a composition and a quantity of the synthetic gas mixture supplied to the microbial digester.

18. The system of claim 17, wherein the means for monitoring includes at least one sensor selected from a group of sensors consisting of:
(a) a first sensor for determining at least one of a quantity and a quality of the synthetic gas mixture generated by the plasma reactor, said first sensor producing a first output signal supplied to the controller;
(b) a second sensor for determining conditions in the microbial digester, said second sensor producing a second output signal that is supplied to the controller;
(c) a third sensor for determining at least one of a quantity and a quality of the chemical product produced by the microbial digester;
(d) a fourth sensor for determining at least one of a quantity and a quality of an effluent exiting the microbial digester; and
(e) a fifth sensor for determining at least one of a quantity and a quality of the synthetic gas mixture that exits the microbial digester with the chemical product.

19. The system of claim 16, further comprising a controller configured to enable a user to choose either a first set of plasma reactor operational parameters that result in production of a first type of chemical product in the microbial digester, or a second set of plasma reactor operational parameters that result in production of a second type of chemical product in the digester.

20. The system of claim 19, wherein the first type of chemical product is ethanol, and the second type of chemical product is acetate.

21. The system of claim 19, wherein the first type of chemical product is a selected higher alcohol, and the second type of chemical product is acetate.

22. The system of claim 16, further comprising a conditioning subsystem to cool the synthetic gas mixture exiting the plasma reactor before the synthetic gas mixture enters the digester, said conditioning subsystem being configured to generate steam for use by the system.

23. The system of claim 16, further comprising a conditioning subsystem to cool the synthetic gas mixture exiting the plasma reactor before it enters the microbial digester, said conditioning subsystem being configured to generate electrical energy for use by the plasma reactor.

24. A system for converting carbon dioxide emissions into a chemical product having a higher value than the carbon dioxide emissions, comprising:
(a) a plasma reactor configured to generate a tailored synthetic gas mixture from the carbon dioxide emissions and a carbonaceous feedstock;
(b) a bio-reactor configured to convert the tailored synthetic gas mixture into the chemical product; and
(c) a controller configured to control the plasma reactor such that a quantity and a composition of the synthetic gas mixture produced by the plasma reactor are as required for enabling the bio-reactor to produce the chemical product.

25. A method for converting carbon dioxide emissions into a chemical product having a higher value than the carbon dioxide emissions and thereby consuming the carbon dioxide emissions, comprising:
(a) introducing the carbon dioxide emissions and a carbonaceous feedstock into a plasma reactor under conditions controlled to generate a synthetic gas mixture;
(b) introducing the synthetic gas mixture into a bio-reactor configured to convert the synthetic gas mixture into the chemical product;
(c) controlling the plasma reactor to modify the synthetic gas mixture being generated based upon at least one of a quality and a quantity of the chemical product being produced by the bio-reactor; and
(d) collecting the chemical product.

26. The method of claim 25, wherein the plasma reactor is controlled such that a quantity and a composition of the synthetic gas mixture produced by the plasma reactor enables the bio-reactor to produce the chemical product.

27. The method of claim 25, further comprising producing a polymer from the chemical product, the polymer acting as a carbon sink to increase an efficiency of consuming the carbon dioxide emissions.

28. The method of claim 25, further comprising capturing CO₂ produced as a byproduct of the method and recycling the CO₂ that is captured through the plasma reactor.
